(19) United States
(12) Patent Application Publication
(10) Pub. No.: US 2025/0339592 A1
(43) Pub. Date: Nov. 6, 2025

(54) BLOOD TREATMENT DEVICE

(71) Applicant: FRESENIUS MEDICAL CARE AG, Bad Homburg (DE)

(72) Inventor: Jenspeter RAU, Bad Homburg (DE)

(73) Assignee: FRESENIUS MEDICAL CARE AG, Bad Homburg (DE)

(21) Appl. No.: 19/100,137

(22) PCT Filed: Jul. 27, 2023
(86) PCT No.: PCT/EP2023/070857

§ 371 Date: Jan. 31, 2025

(30) Foreign Application Priority Data
Aug. 5, 2022 (DE) 10 2022 119 819.7

(51) Int. Cl. A61M 1/16; A61M 1/36; A61M 60/113; A61M 60/279; A61M 60/531

(52) U.S. Cl. CPC A61M 1/1603; A61M 1/3627; A61M 60/113; A61M 60/279; A61M 60/531; A61M 2205/15; A61M 2205/18

(57) ABSTRACT

The invention relates to a blood treatment device 100 comprising or connected to, in each case, at least one extracorporeal blood circuit 400, one pressure sensor 10 for measuring a prevailing fluid pressure in the extracorporeal blood circuit 400, one blood pump 4 for conveying blood through the extracorporeal blood circuit 400, one dialysate circuit 500, one blood leak detector 23 for sensing an escape of blood in the dialysate circuit 500, one blood filter 200, comprising a dialysate chamber 200b, a blood chamber 200a and a semi-permeable membrane 200c separating the two chambers. Moreover comprising at least one interruption means, one control or regulation unit 60, wherein the control or regulation unit 60 is designed to operate the blood pump 4 in a first operating mode and, after detection of a triggering event, to transfer it into a further operating mode in which a conveyance rate of the blood pump 4 is controlled on the basis of a preset or regulated to a target value. Furthermore, the control or regulation unit 60 is designed to activate the interruption means.

### TECHNICAL FIELD

The invention relates to a blood treatment device comprising an extracorporeal blood circuit, a pressure sensor, a blood pump, a blood leak detector, a blood filter, an interruption means, and a control or regulation unit.

### BACKGROUND

In the prior art, various types of blood treatment devices are known. They include, for example, devices for haemodialysis, haemofiltration, ultrafiltration and haemodiafiltration. In the mentioned methods of blood treatment, blood is conducted via an extracorporeal blood circuit by means of a blood pump. In the case of haemodialysis, the blood is purified by a dialyser which has a blood chamber, present in the extracorporeal blood circuit, and a second chamber, especially a dialysate chamber, which chambers are separated from one another by a semi-permeable membrane. During haemodialysis treatment, dialysis fluid flows through the dialysate chamber, with diffusion between the blood and the dialysis fluid causing certain substances to be transported through the membrane and to be removed with the dialysis fluid via a dialysate circuit. In the case of haemofiltration, convection causes certain substances to be filtered from the blood through a semi-permeable membrane. Haemodiafiltration by contrast is a combination of the two methods. In the case of ultrafiltration, dialysis fluid does not flow through the second chamber; instead, water is merely withdrawn from the blood via the semi-permeable membrane. The blood purification treatments of haemodialysis, haemofiltration and haemodiafiltration can be combined with ultrafiltration.

Present in the dialysate circuit is a dialysis fluid pump for conveying the dialysis fluid through the second chamber. An ultrafiltration pump can generate the necessary negative pressure in the dialysis fluid chamber of the dialyser, so that fluid can be removed from the patient to achieve the desired fluid balance.

The semi-permeable membrane of the dialyser usually consists of a multiplicity of capillary walls of hollow fibres, with the blood flowing through the tightly arranged hollow fibres (blood chamber) and the dialysate, which flows through the dialyser, flowing around the blood in the hollow-fibre interspaces (dialysate chamber). The integrity of the semi-permeable membrane ensures separation of blood and dialysate.

Prior to delivery and use during a blood treatment session, the known dialysers are subjected to factory tests in which the integrity of the membrane is checked. A method which has been proven in practice for this purpose provides a bubble point test, involving pressing sterile air into the dialysate chamber, while the blood chamber receives sterile water. Should undesired leaks in the membrane occur, air flows through the membrane and forms bubbles, meaning that the integrity test has failed and the dialyser is discarded. This check for integrity minimizes the risk of capillary breaks and blood leaks, since only those dialysers that have successfully passed the integrity test are considered for use in blood treatment.

Nevertheless. capillary wall breaks and associated blood leaks can occur during a blood treatment session. It is for this reason that common blood treatment devices have various protection systems which protect the patient from a blood leak, which can lead to a hazardous situation for the patient. Such leaks in the membrane cause blood to enter the dialysate from the extracorporeal blood circuit. One protection system known from practice comprises a blood leak detector.

The blood leak detector is conventionally arranged downstream of the dialyser in a line section of the dialysate line, with dialysate flowing through the line during blood treatment and flow-through operation of the blood leak detector thus being carried out and the blood leak detector generating a signal, for example an acoustic or optical alarm, if a predetermined limit characteristic of blood or a blood constituent is exceeded. Such a limit is regularly exceeded if a blood leak occurs, resulting in a response by the protection system, associated with aiming to achieve a safe state which prevents blood loss into the dialysate as far as possible.

Various measures can be initiated to achieve a safe state. Besides the generation of a signal, the blood pump can be stopped. Furthermore, the supply of dialysate into the dialysate chamber can be interrupted. For example, a pump for pumping the dialysate can be stopped or the dialysate side of the dialyser can be bridged by means of a bypass. Furthermore, an ultrafiltration pump can also be used to withdraw water from the blood through a reduction in the pressure on the dialysate side of the dialyser.

It is an object of the present invention to propose a further blood treatment device which prevents increased blood loss into the dialysate.

The object of the invention is achieved by the blood treatment device having the features of claim 1.

Against this background, what is proposed according to the invention is a blood treatment device, wherein the blood treatment device comprises or is connected to, in each case, a blood pump and a venous pressure sensor for the extracorporeal blood circuit, a dialysate circuit and a blood leak detector on its hydraulic side. The extracorporeal blood circuit is not part of the blood treatment device; instead, it is supplemented therewith before the start of a blood treatment session for the purpose of treating the blood of a patient. The extracorporeal blood circuit can be completely or partially provided on a blood cassette or on a blood hose set.

The blood pump is intended for conveying blood through the extracorporeal blood circuit during a blood treatment session when it is connected to the extracorporeal blood circuit and to a blood filter, which can be in the form of a dialyser, said dialyser for its part having a semi-permeable membrane which separates a dialysate chamber and blood chamber present in the dialyser. For example, the blood pump can be in the form of a peristaltic blood pump, and the blood which is transported in a hose can be pumped by the actuators of the peristaltic pump. The blood pump can be arranged upstream of the dialyser. Upstream of the blood pump, a negative pressure can be generated by the blood pump, and upstream of the blood pump, a pressure increased in comparison thereto can be generated.

Furthermore, the blood treatment device comprises at least one interruption means. The at least one interruption means can be suitable for blocking fluid flow in the extracorporeal circuit.

The blood treatment device further comprises a control or regulation unit or is connected to such a unit. The control or regulation unit is designed or programmed to initiate, carry out, control and/or regulate, especially as disclosed herein, specific functions or a method in cooperation with the blood treatment device. For instance, it is designed in particular for activation of the blood pump and at least one interruption means.

Cooperation can be or comprise activation, control or regulation. Cooperation can be or require a signal connection.

In all discussions above and below, the use of the expression “can be” or “can have” and so on is to be understood as synonymous with “is preferably” or “has preferably” and so on and is intended to illustrate an embodiment according to the invention.

Whenever numerical values are mentioned herein, a person skilled in the art will understand them as indicating a numerically lower limit. Provided that this does not lead to a contradiction apparent to a person skilled in the art, a person skilled in the art will therefore always infer, for example, “at least one” when “one” is indicated.

If “programmed” or “designed” is mentioned herein, it is also disclosed that these terms are interchangeable.

Advantageous developments of the present invention are, in each case, subject matter of dependent claims and embodiments.

If an embodiment is mentioned herein, it is an exemplary embodiment according to the invention.

Embodiments according to the invention can comprise one or more of the features mentioned above and/or mentioned in what follows, in any technically possible combination.

In some embodiments of the blood treatment device according to the invention, the control or regulation unit is designed to operate the blood pump in a first operating mode and, after detection of a triggering event, to transfer it into a further operating mode in which a conveyance rate of the blood pump is controlled on the basis of a preset or regulated to a target value.

We have recognized that normally during blood treatment, especially during haemofiltration, haemodiafiltration or ultrafiltration, a pressure gradient from the blood chamber to the second chamber is established for the necessary flow across the semi-permeable membrane and that said pressure gradient is not reduced instantaneously when the blood pump stops. Moreover, we have recognized that, in the case of some blood pump concepts, the pumps have a certain inertia, the result of which is that, when the blood pump stops, an actuator of the blood pump comes to a halt with a time delay. Both aspects can contribute to the continued existence of a transmembrane pressure gradient in the case, for example, of a membrane rupture in the dialyser and to the slow reduction thereof, meaning that further blood can be shifted from the blood chamber into the second chamber despite stoppage of the blood pump. As a result, we have recognized that it may be appropriate to reduce the transmembrane pressure gradient as rapidly as possible and/or to avoid building up a relatively high transmembrane pressure gradient.

In some embodiments, the first operating mode of the blood pump can be a mode in which the blood pump is operated for the purpose of blood treatment in predetermined rates or conveyance rates characteristic of the treatment. Conveyance rates can, characteristically, be in a range between 200 ml/min (millilitres per minute) and 500 ml/min. The further operating mode of the blood pump can be a mode in which the rate or conveyance rate differs from that in the first operating mode in that the blood pump stops or its conveyance rate is reduced.

In some embodiments, a triggering event, as described above, can be detection of blood or a blood constituent in the dialysate circuit, wherein the event is triggered by means of the blood leak detector depending on a determined value and the determined value exceeds a certain threshold.

In some embodiments, the blood leak detector is an optical sensor, wherein said optical sensor comprises a blood leak channel, wherein said blood leak channel in turn monitors the dialysate for the content of blood or the content of a blood constituent. The blood leak detector can detect different transmission behaviours of blood or the blood constituent for, for example, red and green light of a light-emitting diode.

In some embodiments, blood leaks smaller than 0.35 ml/min blood, at an assumed haematocrit value of 32%, are regarded as a non-serious hazardous situation.

In some embodiments, the at least one interruption means is arranged in or on the extracorporeal blood circuit, especially in or on a venous line, or with an effect thereon, wherein the control or regulation unit is designed to activate the interruption means with the goal of counteracting a pressure rise in at least one section of the extracorporeal blood circuit and/or in the blood chamber of the blood filter in the further operating mode of the blood pump in order thus to bring about as effectively as possible a reduction of passage of blood from the blood chamber into the dialysate chamber.

In some embodiments, the interruption means is or comprises a venous hose clamp, a valve or a butterfly valve, wherein the control or regulation unit is designed to activate the interruption means. Here, the interruption means can be closed. The control or regulation unit can be designed to keep the interruption means to be closed in an open state prior to closure, in order to cause the closure according to the invention. The interruption means can be a valve which is arranged downstream of the dialyser. The interruption means can be an actuator which closes a hose line, in which the blood can be conveyed, by compression of the hose wall. As a result, flow of a fluid in the hose can be blocked. The interruption means can be designed such that it is kept open in a flow state and it closes the line in a non-flow state. In other words, the control and regulation unit can generate a signal, by means of which flow through the interruption means is stopped and the interruption means consequently blocks flow in the hose. The control or regulation unit can be designed to activate the interruption means such that it completely or partially closes.

In some embodiments, the control or regulation unit of the blood treatment device according to the invention is designed to activate the interruption means following a moment in which the blood pump has been transferred into the further operating mode, after a predetermined waiting time has been reached or elapsed and/or a venous pressure in the extracorporeal circuit corresponding to a predetermined threshold has been reached or fallen short of. The blood pump can be in the form of a peristaltic pump, especially in the form of a roller pump. Such a roller pump is subject to a moment of inertia when its conveyance rate is reduced or when stoppage occurs, the pump immediately after reduction of its conveyance rate or during the stopping process conveying at a higher rate for a certain period, compared to an expected rate which is established after the moment of inertia has been overcome. This continued conveyance by the blood pump brings about a sudden rise in pressure in at least one section of the extracorporeal blood circuit and/or in the blood chamber of the blood filter if an interruption means were to be closed at the same time as initiating the reduction of the conveyance rate or initiating the stopping process of the blood pump. Such a pressure rise would result in blood continuing to be pushed into the dialysate chamber of the blood filter in the case of capillary breaks or a blood leak.

In some embodiments, the control or regulation unit is programmed to execute the following method during an extracorporeal blood treatment session after detection of blood in the dialysate circuit or after an alarm detected or triggered by the blood leak detector.

The method comprises transfer, especially immediate transfer, of the blood pump from the first operating mode into the further operating mode in which a conveyance rate of the blood pump is controlled on the basis of a preset or regulated to a target value.

In some embodiments, the method comprises the following step. Stopping the blood pump in the further operating mode or reducing its conveyance rate that was produced before transfer into the further operating mode.

In some embodiments, the method initiated by the control or regulation unit comprises determining or establishing whether the predetermined waiting time has been reached or exceeded and/or the pressure in the extracorporeal circuit corresponding to a threshold has been reached or fallen short of.

In some embodiments, the method initiated by the control or regulation unit comprises extending the waiting time if a pressure value exceeding the predetermined threshold is detected at the venous pressure sensor.

In certain embodiments, the blood treatment device comprises at least one dialysate pump for conveying dialysate through the dialysate circuit, which dialysate pump is intended to be arranged in the dialysate side, especially downstream or upstream of the dialysate chamber of the blood filter.

In certain embodiments of the blood treatment device according to the invention, the control or regulation unit is designed, as an alternative or in addition to the above-described embodiments, to operate the dialysate pump in a first operating mode and, after detection of a triggering event, to transfer it into a further operating mode in which a conveyance rate of the dialysate pump is controlled on the basis of a preset or regulated to a target value.

In certain embodiments, the first operating mode of the dialysate pump can be a mode in which the dialysate pump is operated for the purpose of blood treatment in predetermined rates or conveyance rates characteristic of the treatment. The further operating mode of the dialysate pump can be a mode in which the rate or conveyance rate differs from that in the first operating mode.

In certain embodiments, the dialysate pump is in the form of a feed pump, membrane pump or peristaltic pump.

In certain embodiments, the at least one interruption means is arranged in or on the dialysate circuit, especially downstream of the dialysate chamber of the blood filter, or with an effect thereon, wherein the control or regulation unit is designed to activate the interruption means such that the interruption means is closed.

In certain embodiments in a first alternative, the dialysate pump conveys dialysate against the closed interruption means with the goal of bringing about a pressure rise in the dialysate chamber of the blood filter in order thus to build up a counter-pressure or to set a pressure gradient through the semi-permeable membrane, which counter-pressure/pressure gradient is suitable for bringing about back-filtration of the dialysate or the blood-mixed dialysate into the capillaries of the blood chamber of the blood filter. The transmembrane pressure from the dialysate chamber to the blood chamber of the blood filter is positive or at least not negative. An escape of blood through or across the capillaries can thus be prevented or blood which has already escaped can be conducted back into the blood chamber, especially through intact capillaries.

In certain embodiments in a second alternative, the dialysate pump conveys dialysate against the partially closed or open interruption means in the further operating mode with the goal as described above in a certain embodiment in a first alternative. In this case, the conveyance rate is increased in the further operating mode of the dialysate pump; preferably, the dialysate pump is operated at its maximum output or in a range between 600 ml/min and 800 ml/min. Owing to the structure of the blood filter, wherein the cross section perpendicular to the direction of flow of the dialysate in the inlet and outlet is substantially smaller compared to the cross section perpendicular to the direction of flow of the dialysate into the blood filter, what is established at comparably high outputs is a back pressure in the dialysate chamber of the blood filter, since the flow resistance in the outlet, due to the smaller cross section, is higher than in the blood filter.

In some embodiments, the method initiated by the control or regulation unit comprises determining or establishing whether a time or a pressure is within predetermined limits, exceeds or falls short of a limit, exceeds a minimum value and/or does not exceed a maximum value. This can be done on the basis of at least one criterion (limit, range, maximum value, etc.), which, for example, can be stored in a storage device, for instance that of the blood treatment device.

The design according to the invention of the control or regulation unit is based on the fact that it is connected to the relevant components of the blood treatment device and that an algorithm is stored in the control or regulation unit, which algorithm allows the activation according to the invention of the blood pump and the activation according to the invention of the interruption means.

In some embodiments, the blood treatment device is in the form of a dialysis device, haemodialysis device, haemofiltration device or haemodiafiltration device, especially in the form of a device for acute renal replacement therapy, chronic renal replacement therapy or continuous renal replacement therapy (CRRT).

In some embodiments, the blood treatment device is specifically a haemofiltration device. Haemofiltration is a special form of haemodialysis. Here too, the blood is conducted into a specific dialyser, where it is filtered. However, the dialysate and hence mass transfer by diffusion are absent, though convection is maximized. Relatively large quantities of fluid are removed in order to allow detoxification, and this is why they are resupplied to the body in the form of electrolyte solutions. In this connection, the term dialysate is synonymous with provided electrolyte solutions (substitution fluid), which the blood treatment device can supply to the extracorporeal blood circuit by means of, for example, a post-dilution valve. Furthermore, in this connection, the term dialysate circuit is to be understood to mean the hydraulic side or water side of the blood treatment device. The hydraulic system comprises to that effect a supply line for substitution fluid and a discharge line for filtered fluid, especially blood or blood serum. One development of haemofiltration is so-called iso-ultrafiltration, in which haemofiltration as described above is carried out sequentially. Whenever ultrafiltration is mentioned, that includes iso-ultrafiltration.

In some embodiments, the extracorporeal blood circuit is a blood hose set and/or a blood cassette or comprises a blood hose set and/or a blood cassette.

One or more of the advantages mentioned herein can be achievable by means of some embodiments according to the invention, the following being one of said advantages:

The solution according to the invention can advantageously prevent increased passage of blood into the dialysate in the event of blood leaks owing to a capillary break or a rupture in the semi-permeable membrane. The patient therefore does not suffer disadvantageously increased blood loss in the approach according to the invention, and this contributes advantageously to the well-being of the patient and to patient safety.

Further details and advantages of the invention will be apparent from the figures and preferred exemplary embodiments that are discussed below. The blood treatment device according to the invention will be described using the example of a haemodialysis device, but it can also similarly be used in other blood treatment devices, for example a haemodiafiltration device. In the figures:

### BRIEF DESCRIPTION OF THE DRAWINGS

FIG. 1 shows schematically in simplified form a fluid line structure of a blood treatment device according to the invention;

FIG. 2 shows schematically the pressure conditions in the extracorporeal blood circuit during a first operating mode of the blood pump;

FIG. 3 shows schematically the sequence of the method according to the invention.

### DETAILED DESCRIPTION OF AN EXEMPLARY EMBODIMENT

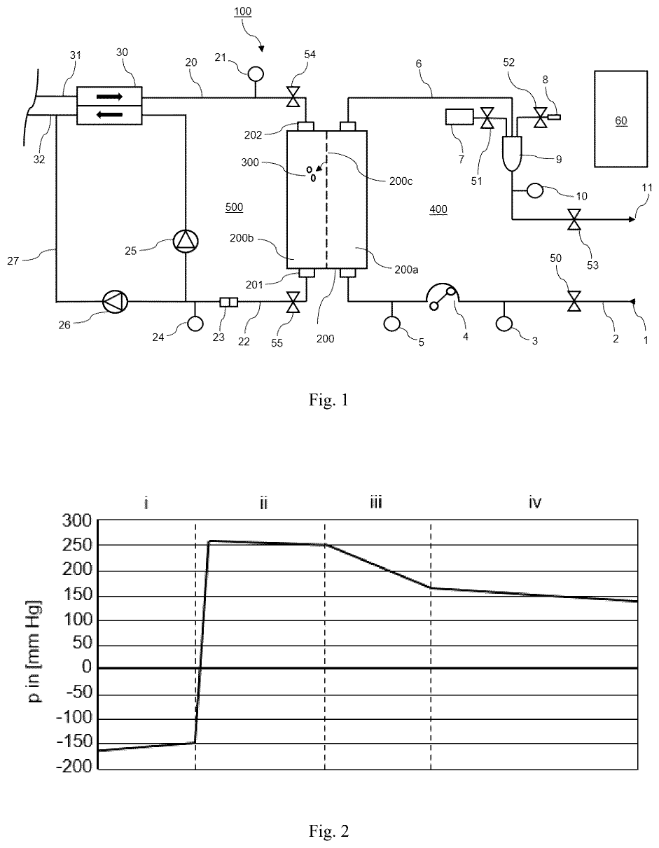

FIG. 1 shows schematically in simplified form a fluid line structure of a blood treatment device 100 according to the invention.

The blood treatment device 100 is connected to an extracorporeal blood circuit 400, which can be connected to the vascular system of the patient, who is not depicted, for treatment by means of double-needle access or by means of single-needle access. Optionally, the blood circuit 400 can, in sections thereof, be present in or on a blood cassette.

The blood circuit 400 comprises an arterial hose clamp 50 as first hose clamp and an arterial connection needle 1 of an arterial line section 2 or is connected thereto. The blood circuit 400 further comprises a venous hose clamp 53 as second hose clamp and a venous connection needle 11 of a venous line section 6 or is connected thereto. An arterial pressure sensor 3 and/or a pre-filter pressure sensor 5 can be provided in the arterial line section 2. Furthermore, a venous pressure sensor 10 and a venous chamber 9, optionally in fluid connection with a venting device 8 and/or with a single-needle chamber 7, can be provided in the venous line section 6. The venting unit 8 and the single-needle chamber 7 are in direct connection with a valve 51;52 each.

Furthermore, the blood treatment device 100 is connected to a dialysate circuit 500 in which dialysate is provided for the treatment. For this purpose, the dialysate circuit 500 comprises a dialysate supply line section 20 into which fresh dialysate can be conducted via the line 31 into the dialysate supply line section 20 by means of, for example, a balance chamber 30. A dialysate inflow pressure sensor 21 and a dialysate inflow valve 54 can be provided in or on the dialysate supply line section 20. Furthermore, the dialysate circuit 500 comprises a dialysate discharge line section 22 into which, by means of the discharge line pump 25 arranged therein, consumed dialysate is conducted into a further line 32 and discarded via the balance chamber 30. Furthermore, the dialysate discharge line section 22 can optionally be fluidically connected to an ultrafiltration line 27, the ultrafiltration pump 26 arranged therein being used to remove excess fluid from the patient and to supply it to the further line 32. The dialysate discharge line section 22 comprises a dialysate outflow valve 55, a blood leak detector 23 and optionally a blood filter outflow pressure sensor 24.

The blood filter 200 comprises the blood chamber 200a connected to the arterial line section 2 and to the venous line section 6. Moreover, the blood filter 200 comprises the dialysate chamber 200b connected to the dialysate supply line section 20 and to the dialysate discharge line section 22. The semi-permeable membrane 200c of the blood filter 200 separates the two chambers from one another.

In the case of haemodialysis, blood of a patient is conducted into the extracorporeal blood circuit 400, into the arterial line section 2 first of all, via the arterial connection needle 1 by means of the blood pump 4 and supplied to the blood chamber 200a of a blood filter 200 in the form of, for example, a dialyser. The substances to be removed pass from the blood into the dialysate by diffusion and/or convection through the semi-permeable membrane 200c, which can define the boundary between the extracorporeal blood circuit 400 and the dialysate circuit 500, with removal of the substances to be removed by the dialysate which flows in the dialysate chamber 200b opposite to the direction of flow of the blood. At the same time, excess fluid from the blood can be removed from the patient via a pressure gradient generatable by the ultrafiltration pump 26 (ultrafiltration). In this case, the transmembrane pressure which is on the semi-permeable membrane 200c over the entire length of the blood filter 200 is always positive or set such that blood plasma or fluid passes from the blood chamber 200a into the dialysate chamber 200b.

The purified blood leaves the blood chamber 200a, is conducted into the venous line section 6 and enters the venous chamber 9, the purified blood lastly being infused into the patient via the venous connection needle 11.

Pumps, actuators, sensors, detectors, hose clamps and/or valves in the region of the blood circuit 400 and the dialysate circuit 500 are connected to the blood treatment device 100 according to the invention or to a control or regulation unit 60 comprised thereby. The control or regulation unit 60 controls, regulates and monitors the blood treatment device 100 and can be in signal connection with each component mentioned herein.

FIG. 2 shows schematically pressure conditions of the extracorporeal blood circuit 400 during a first operating mode of the blood pump 4. The graph is divided into four regions, the regions relating to certain sections of the extracorporeal circuit 400 from the arterial connection needle 1 to the venous connection needle 11. The pressures shown are a snapshot.

Section i relates to a region between the arterial inflow and a pump segment of a hose in which a roller of the blood pump 4 is in contact with the hose. Section ii relates to a region between the blood pump and an inflow to the blood chamber 200a of the blood filter 200. Section iii shows the pressure conditions in the blood filter 200, in particular the pressure profile along the blood filter 200 on its semi-permeable membrane 200c. Section iv relates in turn to the venous line section 6.

The control and regulation unit 60 according to the invention is programmed to control the blood treatment device 100 by means of a method. The method can be executed during a blood treatment session.

Further configurations, programming or designs of the control and regulation unit will be described in what follows in the context of the method according to the invention.

In one exemplary embodiment of the method according to the invention, the patient is first of all in an ongoing blood treatment session, with connection of the arterial connection needle 1 and the venous connection needle 11 to the patient. The blood pump 4 pumps blood at a preset pump rate through the arterial line section 2 into the blood chamber 200a and back into the venous line section 6. At the same time, the venous hose clamp 53 and the arterial hose clamp 50 as interruption means are not closed. Constituents to be removed are withdrawn from the blood by means of the blood filter 200, thereby purifying the blood.

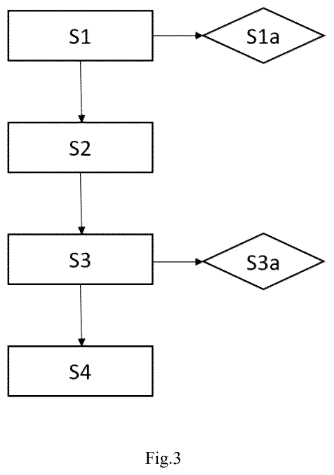

A dialysate flow is set by the balance chamber 30, with an unhindered flow being ensured by the unclosed valves for the dialysate inflow 54 and dialysate outflow 55. Dialysate flows around the blood leak detector 23 downstream of the dialysate chamber 200b, and the blood leak detector 23 monitors the dialysate discharge line section 22 for the presence of blood during the blood treatment session. If there is a blood leak because of breaks in the capillaries or ruptures in the blood filter 200, the blood that has undesirably entered the dialysate chamber 200b reaches the blood leak detector 23. In FIG. 1, blood which has passed through the semi-permeable membrane 200c of the blood filter 200 is identified by the reference sign 300. Such blood is referred to hereinafter as leaked blood 300. For graphical illustration, blood which has leaked in in this way is depicted schematically in a highly simplified manner in particle or droplet form. If the blood leak detector 23 detects leaked blood 300 and if a threshold for the presence of leaked blood 300 is exceeded, the blood leak detector 23 can, as an alternative or in addition to the detection, be suited or programmed to trigger an alarm as a consequence of the detection of leaked blood 300. This leads in turn to initiation or execution of the method according to the invention. FIG. 3 shows schematically the sequence of the method according to the invention. Here, detection of leaked blood 300 can define a triggering event, which is depicted as S1. In addition, this can be associated with optional triggering of an alarm signal, which draws the attention of the treatment personnel to the fact there is a fault. This is shown in S2a.

If leaked blood 300 in the extracorporeal blood circuit 400 has thus been detected or if there is an associated alarm, the control or regulation unit 60 initiates transfer, in particular immediate transfer, of the blood pump 4 from the first operating mode, in which a blood leak did not prevail, into the further operating mode, in which a conveyance rate of the blood pump 4 is controlled or reduced on the basis of a preset or regulated to a target value. In FIG. 3, this step is depicted as S2. In some embodiments, this comprises stopping the blood pump 4 or reducing or immediately reducing its output that was produced immediately before the detection of leaked blood 300. In particularly preferred embodiments, this comprises stopping the blood pump 4.

According to the invention, what happens at the same time or in an overlapping manner is that the interruption means, in particular the arterial hose clamp 50 and the venous hose clamp 53, initially maintain their open state, and thus their state during the operation of the blood pump 4 in its first operating mode, after the blood pump 4 has been transferred into its second operating mode. In a particularly preferred embodiment, the venous hose clamp 53 maintains such a state.

Alternatively or additionally, in some embodiments, the control and regulation unit 60 opens the valve 51 of the single-needle chamber 7 and/or the valve 52 of the venting unit 8 in the event of transfer into a further operating mode of the blood pump 4.

According to the invention, the aim is to compensate for the continued conveyance by the blood pump 4 due to the moment of inertia acting on the pump 4, which continued conveyance otherwise brings about a sudden rise in pressure in at least one section of the extracorporeal blood circuit 400 and/or in the blood chamber 200a of the blood filter 200 if an interruption means were to be closed at the same time as initiating the reduction of the conveyance rate or initiating the stopping process of the blood pump 4. Such a pressure rise would result in increased pushing of blood into the dialysate chamber 200b of the blood filter in the case of capillary breaks or a blood leak. The amount of leaked blood 300, as indicated in FIG. 1, would thus rise.

In a further method step, the respective interruption means maintains its open state until the control or regulation unit 60 initiates, lastly, activation of the interruption means following a moment in which the blood pump 4 has been transferred into the further operating mode, after a predetermined waiting time has been reached or elapsed and/or a venous pressure in the extracorporeal circuit 400 corresponding to a predetermined threshold has been reached or fallen short of. This in fact describes a time delay before the actual closure of the interruption means and is designated as step S3 in FIG. 3. Mechanical actuators and/or solenoid valves can be provided for this purpose. To realize the activation of various interruption means, various designs are available to a person skilled in the art, and so details will not be provided here.

The duration of the waiting time is dependent on the pump type, on the previously set conveyance rate, on the previously set transmembrane pressure, and on the treatment type selected for the blood treatment session. FIG. 2 shows, for a blood pump conveyance rate of 250 ml/min, characteristic pressure conditions in the extracorporeal blood circuit 400 during haemodialysis treatment. A venous pressure sensor 10 is arranged in a region which represents region iv in FIG. 2. The pressure profile in regions i to iv can vary for a haemodiafiltration treatment, depending on whether a substitution fluid is introduced into the extracorporeal blood circuit 400 into a line section upstream of the blood filter 200 (pre-dilution) or into a line section downstream of the blood filter 200 (post-dilution).

If the blood pump 4 is preferably stopped in the further operating mode, the pressure profile approaches 0 mm Hg in region iv during the stopping process. If the blood pump 4 comes to a complete halt, a pressure which tends towards 0 mm Hg or is 0 mm Hg or represents a static pressure value of the blood in the hose or hose section is established at the venous pressure sensor 10. Depending on the type of venous pressure sensor 10 to be used, the resolution of the determined pressure value is linked to limits typical for the design. The determined pressure value can thus deviate from the actual pressure value, for example by 10-20 mm Hg.

The waiting time is thus based on the period of time required by the blood pump 4 to come to a complete halt after transfer into the second operating mode. To this end, at least one value which, for example, was determined by testing can be stored in a storage device of the blood treatment device 100. Furthermore, as an alternative, a fixed time value can be stored or the blood treatment device 100 can have a computation unit which calculates the time value for the waiting time on the basis of the present conditions for the blood treatment session. The control and regulation unit 60 is in signal connection with the computation unit and is suitable for operating the blood treatment device 100 on the basis of the calculated value.

In some embodiments, the control or regulation unit 60 can be designed to extend the waiting time if a pressure value exceeding the predetermined threshold is detected at the venous pressure sensor 10. Such an optional step is designated as S3a in FIG. 3.

In some embodiments, as an alternative to waiting time, the already described pressure values which are measured at the venous pressure sensor 10 are used as the starting point for the activation of the interruption means or plurality of interruption means. The at least one pressure value required therefor can be stored in the storage device of the blood treatment device 100.

In a further method step, the interruption means is controlled such that it is closed after it has been established that the predetermined waiting time has been reached and/or after it has been established that the pressure in the extracorporeal circuit corresponding to a threshold has been reached or fallen short of. Such closure of an interruption means is designated as step 4 in FIG. 3. In a particularly preferred embodiment, the venous hose clamp 53 is closed. Alternatively or additionally, in some embodiments, the valve 51 on the single-needle chamber 7 and/or the valve 52 on the venting unit 8 are closed if they were previously opened in another method step.

As an alternative or in addition to the above-mentioned exemplary embodiment, in a first alternative, the control or regulation unit 60 in certain embodiments can be programmed such that, when the blood pump 4 has been transferred into the further operating mode, the dialysate outflow valve 55 in the dialysate circuit 500 as interruption means is activated and consequently closed or partially closed, the balance chamber 30 being set in such a way to allow dialysate flow against the dialysate outflow valve 55. Optionally, a further pump can be provided in the dialysate supply line section 20 for this purpose.

In a second alternative, the control and regulation unit 60 in certain embodiments can be programmed such that, when the blood pump 4 has been transferred into the further operating mode, a dialysate in the dialysate circuit 500 in a further operating mode is conducted at a higher rate through the dialysate chamber 200b by means of the balance chamber 30, the flow pump 25 and/or the ultrafiltration pump 26. Owing to the structure of the blood filter 200, wherein the cross section perpendicular to the direction of flow of the dialysate in the inlet 202 and outlet 201 is substantially smaller compared to the cross section perpendicular to the direction of flow of the dialysate into the blood filter 200, what is established at comparably high outputs is a back pressure in the dialysate chamber 200b of the blood filter 200, since the flow resistance in the outlet 201, due to the smaller cross section, is higher than in the blood filter 200. The cross-sectional area of the blood filter 200 can be at least two to four-times greater than the cross-sectional area of the outlet 201, depending on the design or the model of the blood filter 200.

Therefore, in the case of the first and the second alternative in certain embodiments, what builds up in the dialysate chamber 200b is a pressure suitable for conducting fluid from the dialysate chamber 200b across the semi-permeable membrane 200c into the blood chamber 200a. In particular, leaked blood 300 can be conducted into the blood chamber 200a. Optionally, at the same time, the blood pump 4 can continue to convey blood or the blood/dialysate mixture, so that the blood or the blood/dialysate mixture does not come to a halt and coagulation of the blood is advantageously avoided. The control and regulation unit 60 operates the blood pump 4 such that the pressure in the blood chamber 200a is not greater than the pressure in the dialysate chamber 200b. Therefore, a neutral or negative transmembrane pressure is established in the blood filter 200. The transmembrane pressure can be evaluated by means of an evaluation unit of the blood treatment device 100 on the basis of the direct measurement of the volume at the dialysate inflow pressure sensor 21 and dialysate outflow pressure sensor 24, the control and regulation unit 60 being programmed to change the dialysis flow volume in such a way that a positive transmembrane pressure is not established. Moreover, the transmembrane pressure can be measured in various ways and the existence thereof can be directly or indirectly established. To this end, various designs are available to a person skilled in the art, and so details will not be provided here.

1. A blood treatment device comprising or connected to, in each case, at least
one extracorporeal blood circuit;
one pressure sensor for measuring a prevailing fluid pressure in the extracorporeal blood circuit;
one peristaltic blood pump for conveying blood through the extracorporeal blood circuit;
one blood filter, comprising a dialysate chamber and a blood chamber;
at least one interruption means, suitable for blocking flow in the extracorporeal blood circuit;
one control or regulation unit, wherein the control or regulation unit is designed to operate the blood pump in a first operating mode and, after detection of a triggering event, to transfer it into a further operating mode, and
the control or regulation unit is further designed to activate the interruption means, wherein the interruption means consequently blocks the flow according to a preset with a time delay for transfer of the blood pump into the further operating mode.

2. The blood treatment device according to claim 1, wherein the pressure sensor is a venous pressure sensor or comprises a venous pressure sensor.

3. The blood treatment device according to claim 1, wherein the control or regulation unit is further designed such that the interruption means is activated following a moment in which the blood pump has been transferred into the further operating mode, after a predetermined waiting time has been reached or elapsed and/or a venous pressure in the extracorporeal circuit corresponding to a predetermined threshold has been reached or fallen short of and/or a transmembrane pressure corresponding to a predetermined threshold has been reached or fallen short of.

4. The blood treatment device according to claim 1, wherein the control or regulation unit is designed to extend the waiting time if a pressure value exceeding the predetermined threshold is detected at the venous pressure sensor.

5. The blood treatment device according to claim 1, wherein the control or regulation unit is designed to stop the blood pump in the further operating mode or to reduce its conveyance rate that was produced before transfer into the further operating mode.

6. The blood treatment device according to claim 1, wherein the interruption means is arranged in or on the extracorporeal blood circuit.

7. The blood treatment device according to claim 1, wherein the interruption means is or comprises a venous hose clamp, a valve or a butterfly valve, and wherein the control or regulation unit is designed to activate the interruption means such that it is closed.

8. The blood treatment device according to claim 1, wherein the blood treatment device comprises a blood leak detector for sensing an escape of blood.

9. The blood treatment device according to claim 1, wherein the blood treatment device comprises a dialysate circuit and the blood leak detector is arranged in the dialysate circuit, especially in a line section downstream of the dialysate chamber of the blood filter.

10. The blood treatment device according to claim 1, wherein the triggering event is detection of blood in the dialysate circuit, wherein the event is triggered by means of the blood leak detector depending on a determined value and the determined value exceeds a certain threshold.

11. The blood treatment device according to claim 1, wherein the blood leak detector 23 is an optical or acoustic sensor.

12. The blood treatment device according to claim 1, wherein the control or regulation unit is programmed to execute a method for controlling a blood treatment device during a blood treatment session, wherein the method comprises the following steps effected after detection of blood or after an alarm detected or triggered by the blood leak detector:
transferring the blood pump from the first operating mode into the further operating mode, in which a conveyance rate of the blood pump is reduced;
establishing that the predetermined waiting time has been reached and/or establishing that the pressure in the extracorporeal circuit corresponding to a threshold has been reached or fallen short of;
activating the interruption means such that it is closed.

13. The blood treatment device according to claim 12, further comprising the step of:
stopping the blood pump in the further operating mode or reducing its conveyance rate that was produced immediately before transfer into the further operating mode.

14. The blood treatment device according to claim 12, further comprising the step of:
extending the waiting time if a pressure value exceeding the predetermined threshold is detected at the venous pressure sensor.

15. The blood treatment device according to claim 12, wherein the control or regulation unit is programmed to initiate, in any combination, some or all of the steps.

16. The blood treatment device according to claim 1, wherein the blood treatment device is in the form of a dialysis device, haemodialysis device.

17. The blood treatment device according to claim 1, wherein the blood treatment device is in the form of a device for acute renal replacement therapy, chronic renal replacement therapy or continuous renal replacement therapy.

18. The blood treatment device according to claim 1, wherein the interruption means is arranged in or on a venous line, or with an effect thereon.
